(19) United States
(12) Patent Application Publication
(10) Pub. No.: US 2008/0114228 A1
(43) Pub. Date: May 15, 2008

(54) Method Of Manufacturing An Auto-Calibrating Sensor

(76) Inventors: Joseph McCluskey, Sharon, MA (US); Alun Griffith, Inverness-shi (GB); Grenville Robinson, Ross-shire (GB); Gordon Spalding, Inverness-shire (GB); David Taylor, Inverness-shire (GB); Erica Mary Beck, Nairn (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/574,336

(22) PCT Filed: Aug. 31, 2005
(86) PCT No.: PCT/US05/31286

§ 371 Date: Jul. 19, 2007

Related U.S. Application Data
(60) Provisional application No. 60/606,334, filed on Aug. 31, 2004.

(51) Int. Cl. A61B 5/145; B05D 5/12; G08B 1/08; G01N 33/66; G01N 27/00

(52) U.S. Cl. 600/365; 427/58; 340/539.12

(57) ABSTRACT

The invention concerns a sensor that, when exposed to a fluid, develops a measurable characteristic that is a function of the level of an analyte in the fluid and of a calibration quantity of the sensor. A calibration quantity is some physical, chemical or other inherent property that the sensor possesses that affects its response to the analyte. The sensor includes an RFID tag that receives, stores and conveys information representing the calibration quantity. The wireless device is incorporated into or attached to the sensor during the manufacturing process and before the sensor is calibrated. The wireless device can be written wirelessly once the calibration has been done. This does not involve any additional handling of the sensor and can be done once the sensor has been placed into a protective enclosure. Because of this, the process of wirelessly transmitting the calibration information to the wireless device does not alter any pre-existing calibration quantities and neither does it introduce any new calibration quantities, thus preserving the calibration of the sensor even though the sensor has been wirelessly modified to carry information representing its calibration quantity.

### PRIORITY

This application claims priority benefits under 35 U.S.C. §§ 120 and 371 of International Application PCT/US2005/031286 filed on 31 Aug. 2005, which claims priority benefits to U.S. Provisional Application Ser. No. 60/606,334 filed on 31 Aug. 2004, which both applications are hereby incorporated by reference in their entireties into this application.

### FIELD OF THE INVENTION

The invention relates to an auto-calibrating sensor for use, in healthcare management, law-enforcement, dope-testing, sanitation or otherwise, for measuring the concentration of any analyte, such as glucose, lactate, urate, alcohol, therapeutic drugs, recreational drugs, performance-enhancing drugs, biomarkers indicative of diseased conditions, hormones, antibodies, metabolites of any of the aforesaid, combinations of any of the aforesaid, other similar indicators or any other analyte in a fluid, especially a physiological fluid such as blood, interstitial fluid (ISF) or urine. Much of the following discussion will concentrate upon the use of such a sensor for the purpose of blood glucose measurement and control but the principles discussed are much more widely applicable; indeed, they are applicable to the detection of any analyte in any fluid.

### BACKGROUND OF THE INVENTION

Glucose monitoring is a fact of everyday life for diabetic individuals. The accuracy of such monitoring may have significant impact on the quality of life. Generally, a diabetic patient measures blood glucose levels several times a day to monitor and control blood sugar levels. Failure to control blood glucose levels within a recommended range can result in serious healthcare complications such as limb amputation and blindness. Furthermore, failure to accurately measure blood glucose levels may result in hypoglycaemia. Under such conditions the diabetic patient may initially enter a comatose state, and if untreated may die. Therefore, it is important that accurate and regular measurements of blood glucose levels are performed.

People suffering from diabetes are often at a higher risk of other diseases. Diabetes also contributes to kidney disease, which occurs when the kidneys do not filter properly and protein leaks into urine in excessive amounts, which eventually can cause kidney failure. Diabetes is a cause of damage to the retina at the back of the eye and also increases risk of cataracts and glaucoma. Nerve damage caused by diabetes may interfere with the ability to sense pain and contributes to serious infections. A number of glucose meters are currently available which permit an individual to test the glucose level in a small sample of body fluid.

Many of the glucose meter designs currently available make use of a disposable test sensor, e.g. a strip, which in combination with the meter, electrochemically or photometerically measures the amount of glucose in the blood sample. To use these meters, the user first punctures a finger or other body part using a lancet to produce a small sample of blood or interstitial fluid. The sample is then transferred to a disposable test strip. The test strips are typically held in packaging containers or vials prior to use. Generally, test strips are quite small and the sample receiving area is even smaller. Usually, the disposable strip is inserted into a meter through a port in the meter housing prior to performing a test for an analyte in body fluids such as blood, ISF or urine etc.

The variation in the manufacturing process and chemistry of the strips causes them to need to have calibration coefficients or codes assigned to them so that their performance is mathematically correlated to a specific defined performance curve. Some examples of process and chemical variations will be described later, but for now it is sufficient to note that these variations result in sensors having different physical, chemical or other inherent properties that affect the way they respond to an analyte. Thus, different sensors will respond slightly differently to the same concentration of analyte in a fluid. Because they respond differently, their response must then be adjusted by an amount that is determined by calibration. The calibration process allows one to determine one or more adjustment coefficients that, when applied to the response of the sensor, will normalize it to a predefined standard. To help us to refer to the physical, chemical or other inherent characteristics of the sensor, we have coined the expression “calibration quantity” and we shall use it from now on. A calibration quantity is some property that the sensor possesses that affects its response. It may be a single property, such as sensitivity; it may be a combination of many, such as sensitivity, non-linearity, hysteresis, etc. It may be some structural property such as size that contributes to its response behaviour, either by affecting other calibration quantities like sensitivity, or by making an individual contribution. All of these things, alone or together, are calibration quantities, from which it can be seen that the term denotes a broad class. It is to be distinguished from the one or more adjustment coefficients that are derived from the calibration process and, when applied to the response of the strip, will normalize it to a predefined standard. These coefficients are shorthand representations of calibration quantities; they are information representing the calibration quantities, but they are not the calibration quantities themselves, which are real properties of the sensors. Thus, where we wish to refer to the adjustment coefficients or any other information representing them, and therefore representing the calibration quantities of the sensors, for example a code pointing to a location in a look-up table at which the relevant adjustment coefficients may be found, we use the expression “information representing the calibration quantity.” The distinction is a simple one, but it is worth setting out here for the avoidance of doubt.

When using a strip to which a calibration coefficient or code has been assigned, a diabetic patient typically has to read calibration data printed on a vial containing the sensor, enter it into the blood glucose monitoring system and confirm it for each test. The test strip is then inserted in the blood glucose monitoring system.

This can be undesirable since it can take time for a user to learn proper use of the process involved in diabetes testing and errors of operation by a user can occur. It is also undesirable since a user may be put off by tedious repetitive action of inserting calibration codes into a blood glucose monitoring system, which reduces the accuracy of glucose levels and can lead to complicated health conditions. It is further undesirable since repetitive testing on a localised area results in lack of feeling especially around the finger tips (nerve damage) and calluses can form making operation of the buttons difficult. This creates a problem for diabetics as technology pushes miniaturisation to new limits, partially driven by the need to make blood glucose meter systems acceptable and not ‘out of place’ i.e. to make the diabetic patient to feel as ‘normal’ as possible. Users can also have difficulty in using such devices because of the resultant effects of their medical conditions again causing difficulty, entering data via buttons or keypads etc.

Another problem with the insertion of calibration codes is again that long term diabetes sufferers who have not managed to fully control their illness may be suffering from cataracts or glaucoma. Such illnesses make the use and operation of blood glucose meter systems problematic with partially sighted sufferers, for whom basic testing could be considered an achievement, let alone inputting of calibration codes into a blood glucose meter.

Another problem with the insertion of calibration codes is that blood glucose testing is a time consuming affair. Typically each test can take up to five minutes which includes washing of hands, inserting a strip in blood glucose meter, lancing the finger and drawing blood, applying the blood onto the strip, inputting the batch specific calibration code, and waiting and reading the glucose level produced by the blood glucose meter. Typically, diabetics are recommended to test their glucose levels around four times a day and they often need to be encouraged to test themselves. Performing time consuming manual steps potentially minimises the frequency a diabetic tests himself and can lead to a downward spiral for the user e.g., lack of testing resulting in further complications which in turn discourages a diabetic from testing further, for example because of the need to lance and enter calibration strip data into a blood glucose meter.

The confirmation of test calibration data on a display such as an LCD display and/or LED display can also lead to problems for users of all ages and users of all levels of diabetes. During pre-breakfast testing a diabetic may have difficulty focusing on such a small display and could enter an incorrect calibration code. Similarly, a conscientious diabetic wishing to test himself at the post evening meal or pre-bed time may be tired and feeling drowsy and may inadvertently input the incorrect calibration code into the blood glucose meter. Again, this could lead to complicated health conditions especially where a diabetic is about to sleep for the night thinking his glucose level is normal when in actual fact he may be entering an unconscious state because he is in a hypoglyeaemic condition.

Also, if a diabetic does enter into a hypoglycaemic condition and is found by his partner or care giver, then it would further cause confusion if the care giver is not trained in glucose testing. The caregiver could summon help or alternatively use the meter to test the glucose level him/herself. The care giver may not however, be aware that a manual cumbersome calibration code needs to be inputted into the blood glucose meter before testing, resulting in an incorrect calibration code being inputted leading to further complications.

Similarly, since test strips are small in size, visually-impaired diabetics have difficulty in knowing how many test strips are left in a vial. This can be a problem to diabetics especially when they leave their normal surroundings for a length of time e.g. travelling away on a whim, on holiday etc. and could potentially leave them without enough test strips for the duration of their time away from home. Not only is this potentially dangerous to a diabetic, but also is inconvenient. It would therefore be beneficial to a diabetic especially a partially sighted diabetic that an audio and/or visual means was provided on a blood glucose meter system which automatically informs a user of the number of strips remaining in a vial.

Many modern industries and in particular the diabetes monitoring industry are therefore presented with the challenge of providing a metering system which is able to allow a user to use such a system without the need to enter calibration codes. Another challenge facing the diabetes monitoring industry is the use of monitoring devices by people with disabilities.

We have considered the possibility of simply attaching the calibration information to the sensors in machine-readable form—and one example of how this might be done is by attaching a barcode label—and providing the monitoring device with a device capable of reading the information, such as a barcode reader. On the face of it, this solves the problems outlined above: the monitoring device simply reads the calibration information off the sensor when it is inserted, and uses that information to normalize the response of the sensor.

But it does not work, and the reasons why it does not work are not immediately apparent, so we shall explain them.

The principal contributor to the variations in the response from different sensors, which we recall are attributable to the calibration quantities of the sensors, is the existence of tolerances and variations in the manufacturing process. When we use the expression “tolerances and variations” we are, of course speaking of small effects, indeed effects so small that it may be uneconomical to engineer them out of the manufacturing process; hence the need for calibration in the first place. These small effects are large enough to upset the accuracy of blood glucose measurements and indeed the accuracy of any analyte measurement where a certain level of accuracy is needed. So the sensors are calibrated and the calibration information is recorded.

Now consider the process of applying barcode labels with the calibration information on them to the sensors. We have been speaking hitherto of processes that have been so finely engineered that only small variations and tolerances remain that may be uneconomic to engineer out and have described how these small variations lead to different calibration quantities, and hence different calibration information, for the sensors. But now we are speaking of a process that is very difficult if not impossible to engineer down to the same level of tolerances. The step of attaching a barcode label involves the application of pressure and the use of an adhesive that may out-gas contaminants. In short, it is a process that will either alter the pre-existing calibration quantities of the sensors or it will introduce new calibration quantities, such as dimensions owing to the application of pressure, or chemical properties owing to the introduction of contaminants. In any case, the altered or new calibration quantities will no longer be properly represented by the calibration information that was previously printed on the label, which in turn means that the sensor must be recalibrated. So one is back to square one, except that one now has a label attached to the sensor with the wrong calibration information on it.

### SUMMARY OF THE INVENTION

The present invention is designed to address the problems outlined above. Whilst those problems have been described particularly with reference to the management of diabetes, where accuracy is absolutely essential and the abilities of the user may be impaired, we nonetheless regard the problem as more general. Indeed, if one is testing any fluid for any analyte using a sensor that is to be exposed to the fluid, where the degree of accuracy required leads to calibration, and one wishes to avoid the inconvenience of inputting calibration information, coefficients or codes, the present invention will be of considerable assistance.

Our solution is to use, on the sensor, of a wireless device into which the calibration information, i.e. the information representing the calibration quantity of the sensor, can be wirelessly written. Crucially, in the present invention, the wireless device may be incorporated into or attached to the sensor during the manufacturing process and before the sensor is calibrated. Equally crucially, the wireless device is written to wirelessly once the calibration has been done. This does not involve any additional handling of the sensor and indeed at can be done once the sensor has been placed into a protective enclosure. Because of this, the process of wirelessly transmitting the calibration information to the wireless device does not alter any pre-existing calibration quantities and neither does it introduce any new calibration quantities.

Therefore, one aspect of the present invention is that it involves a method of manufacturing a sensor that, when exposed to a fluid, develops a measurable characteristic that is a function of the level of an analyte in the fluid and of a calibration quantity of the sensor, and has a wireless device adapted to receive, store and convey information representing the calibration quantity, the method comprising:

at least partly manufacturing the sensor so that it possesses the calibration quantity and includes the wireless device;

then wirelessly transmitting the information representing the calibration quantity to the wireless device; and

then, optionally, completing the manufacture of the sensor.

It will be noted that one aspect of the present invention therefore requires sufficient manufacturing steps to be performed, before the information representing the calibration quantity is transmitted to the wireless device, for the calibration quantity of the sensor to be determined. Subsequent steps may be performed, and we would not wish to exclude that possibility, so long as they do not affect the calibration. The earliest point in the manufacturing process at which the calibration and transmission can take place can easily be determined by trial and error—if subsequent steps affect the calibration, it has been done too early.

Another aspect of the present invention is that it involves a method of calibrating a sensor that, when exposed to a fluid, develops a measurable characteristic that is a function of the level of an analyte in the fluid and of a calibration quantity of the sensor, and incorporates a wireless device adapted to receive, store and convey information representing the calibration quantity, the method comprising wirelessly transmitting the information representing the calibration quantity to the wireless device incorporated in the sensor.

An alternative aspect of the invention is that it involves a method of manufacturing a sensor that, when exposed to a fluid, develops a measurable characteristic that is a function of the level of an analyte in the fluid and of a calibration quantity of the sensor, and has a wireless device adapted to receive, store and convey information representing the calibration quantity, the method comprising: completing the manufacture of the sensor so that it possesses the calibration quantity and includes the wireless device; and then wirelessly transmitting the information representing the calibration quantity to the wireless device.

The present invention finds application to a variety of sensors, including photometric or colorimetric sensors, where the measurable characteristic may be an opacity, a transparency, a fluorescence intensity, a transmissivity, a reflectivity, an absorptivity or an emissivity, a transmission, reflection, absorption, emission or excitation spectrum, peak, gradient or ratio, any one of more parts of such a spectrum, a colour, an emission polarization, an excited state lifetime, a quenching of fluorescence, a change over time of any of the aforesaid, any combination of the aforesaid, or any other indicator of the extent to which exposure of the sensor to the fluid affects its optical characteristics.

Typical photometric or calorimetric sensor comprises a substrate and at least a first reagent. The reagent may include a catalyst and a dye or dye precursor, where the catalyst catalyses, in the presence of the analyte, the denaturing of the dye or the conversion of the dye precursor into a dye. In the field of glucose monitoring, the catalyst may be a combination of glucose oxidase and horseradish peroxidase with the reagent including a leuco-dye (a reduced dye precursor). Suitable leuco-dyes are 2,2-azino-di-[3-ethylbenzthiazoline-sulfonate], tetramethylbenzidine-hydrochloride and 3-methyl-2-benzothiazoline-hydrazone in conjunction with 3-dimethylamino-benzoicacide.

As already discussed, the group of analytes to which the present invention may be applied is large and includes, in addition to glucose, HbA1C, lactate, cholesterol, alcohol, ketones, urate, therapeutic drugs, recreational drugs, performance-enhancing drugs, biomarkers indicative of diseased conditions, hormones, antibodies, metabolites of any of the aforesaid, combinations of any of the aforesaid, or other similar indicators.

These photometric or colorimetric sensors may be at least partly manufacturing by positioning a reagent film or membrane over a opening in a substrate (for a sensor that relies on measuring transmitted light), positioning a reagent film or membrane over a portion of a substrate (for a sensor that relies on measuring transmitted or reflected light) or placing a reagent in a chamber in a substrate (again, for a sensor that relies on measuring transmitted or reflected light). At this point or later, the wireless device may be attached to the substrate. Either then or subsequently the information representing the calibration quantity is transmitted to the wireless device.

The present invention is also applicable to electrochemical sensors comprising electrodes, where the measurable characteristic is an inter-electrode impedance, an inter-electrode current, a potential difference, an amount of charge, a change over time of any of the aforesaid, any combination of the aforesaid or any other indicator of the amount of electricity passing from one electrode to another, or the extent to which exposure of the sensor to the fluid generates electrical energy or electrical charge or otherwise affects the electrical characteristics of the sensor.

Typical electrochemical sensors comprises a substrate, an electrode layer containing the electrodes, and at least a first reagent layer. These sensors may be at least partly manufactured by depositing an electrode layer containing the electrodes on a substrate and depositing a reagent layer on the substrate and optionally over the electrode layer. When the analyte is glucose, the reagent layer optionally includes glucose oxidase.

In the case of electrochemical sensors, the method of manufacture may comprise depositing a component of the wireless device, especially depositing it in the electrode layer. This component may be an antenna, either a coil or a micro-strip antenna, but if it is a micro-strip antenna, the electrodes in the electrode layer may themselves form the antenna. We believe this to be a new and useful idea in itself irrespective of the calibration of the sensor, since the wireless device could be used to carry additional or alternative information.

Therefore, a third aspect of the present invention is that it involves an electrochemical sensor comprising:

a substrate;

an electrode layer containing electrodes; and

at least a first reagent layer;

the sensor being so configured that, when exposed to a fluid, it develops a measurable electrical characteristic that is a function of the level of an analyte in the fluid;

the sensor further comprising a wireless device adapted to receive, store and convey information, including a micro-strip antenna formed by the electrodes in the electrode layer.

Returning to the method of manufacture, it will then include affixing remaining components of the wireless device to the sensor, in electrical contact with the deposited component, before the information representing the calibration quantity is transmitted to it.

An insulation layer may be deposited over the electrode layer and the reagent layer over the insulation layer, the insulation layer preventing contact between the electrodes and the reagent layer otherwise than at one or more selected contact zones. This standardizes the internals of the sensor, ensuring that the calibration quantities of different sensors are closely related.

A second reagent layer may be deposited over the first reagent layer, for example an electron transfer mediator such as ferricyanide.

The deposition of at least one layer can be achieved by means of a printing process such as screen printing, ink jet printing, lithography, flexography, gravure, rotogravure, laser marking, slot/die coating or spray coating. Cylinder screen printing is quite suitable.

In the interests of greater efficiency, a plurality of sensors may be manufactured in a batch, especially in a batch on a single substrate. Optionally, they are manufactured in a continuous process, especially on a continuous web of substrate.

This process may involve continuously passing the continuous web through an electrode deposition station and a reagent deposition station, at the electrode deposition station, depositing electrode layers containing the electrodes of respective sensors (and possibly a component such as a micro-strip antenna of the wireless device), and at the reagent deposition station, depositing reagent layers of respective sensors over the electrode layers. It may also include continuously passing the continuous web through an insulation deposition station, at the insulation deposition station, depositing insulation layers of respective sensors over the electrode layers and at the reagent deposition station, depositing reagent layers of respective sensors over the insulation layers, the insulation layers preventing contact between the electrodes and the reagent layers otherwise than at selected contact zones. It may also include continuously passing the continuous web through a second reagent deposition station, and at the second reagent deposition station, depositing a second reagent layer of respective sensors over the first reagent layers.

Subsequently, the continuous web may be continuously passed through a wireless device fixing station, at which a wireless device is fixed to respective sensors. The web may then be cut into ribbons, each ribbon containing a plurality of sensors.

When sensors are batch-manufactured, in either a flat-bed or staged process or in a continuous process, information representing the same calibration quantity may be transmitted to the wireless devices of a plurality of sensors at once or virtually simultaneously. In particular, a plurality of sensors may be placed into a protective enclosure and then information representing the same calibration quantity may be wirelessly transmitted to the wireless devices of those plurality of sensors at once or virtually simultaneously. This saves time and ensures the sensors are handled to the minimum degree possible.

This invention also extends to a sensor that, when exposed to a fluid, develops a measurable characteristic that is a function of the level of an analyte in the fluid and of a calibration quantity of the sensor, and has a wireless device adapted to receive, store and convey information representing the calibration quantity, in which the wireless device contains information representing the calibration quantity of the sensor.

Wireless communication at radio frequencies is suitable, as it is unlikely to cause heating of the sensor, which may change its calibration quantity. Thus, for the wireless device, an RFID tag is suitable for use with, for example ISO 14443 or ISO 15693, on a frequency of 13.56 MHz or 2.45 GHz.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention (wherein like numerals represent like elements), of which:

FIG. 1 shows a schematic plan view of a single use test strip for receiving a patient's blood, according to a first exemplary embodiment of the invention having an RFID tag integrated thereon.

FIG. 2 shows a schematic plan view of a single use test strip for receiving a patient's blood and a blood glucose meter, according to a further exemplary embodiment of the invention having an RFID tag integrated on the single use test strip having conductive tracks feeding to an edge of the test strip.

FIG. 3 shows a schematic plan view of a single use test strip for receiving a patient's blood and a blood glucose meter, according to a further exemplary embodiment of the invention having an RFID tag integrated on the single use test strip. The RFID tag is written to by RF means during the manufacturing stage of the single use test strip.

FIG. 4 shows a schematic plan view of a multi use test strip or module in the form of a disc for receiving a patient's blood, according to a further exemplary embodiment of the invention having an RFID integrated thereon.

FIG. 5 shows a system diagram depicting a system for extracting and monitoring a bodily fluid sample according to a further exemplary embodiment of the invention within which, for example, the embodiments of FIG. 4 or FIG. 5 can be used.

FIG. 6 shows a schematic plan view of a packaging container such as a plastic or cardboard box according to an alternative aspect of the invention containing a blood glucose meter, a vial containing strips, a lancing device, a container containing control solution, and an instruction guide. An RFID tag containing batch information such as product expiry date and/or country of import/export, and/or helpline information, and/or manufacturer, and/or conditions of use such as environmental or physiological limitations is attached to the packaging container.

FIG. 7 shows a table of information which may be loaded from a RFID tag to the meter and from the meter to the RFID tag in accordance with example embodiments of the present invention.

FIG. 8 shows a schematic perspective view of a vial having an RFID tag integrated thereon.

FIG. 9 shows a base member for a test strip;

FIG. 10 shows the layout of carbon tracks applied to the base member;

FIG. 11 shows the layer of insulation applied to the strip;

FIG. 12 shows the enzyme reagent layer;

FIG. 13 shows an adhesive layer;

FIG. 14 shows a layer of hydrophilic film;

FIG. 15 shows the cover layer of the strip;

FIGS. 16A and 16B show two alternative deposition patterns useful in manufacturing strips in a continuous process;

FIGS. 17A and 17B show an exemplary electrochemical sensor which can be manufactured using the continuous method;

FIG. 18 shows a schematic view of an apparatus for practising the continuous manufacturing method;

FIG. 19 shows post-processing of a web printed with sensors to produce sensor ribbons.

FIGS. 20A and 20B show a further alternative embodiment of a sensor which can be manufactured using the continuous manufacturing method.

### DETAILED DESCRIPTION

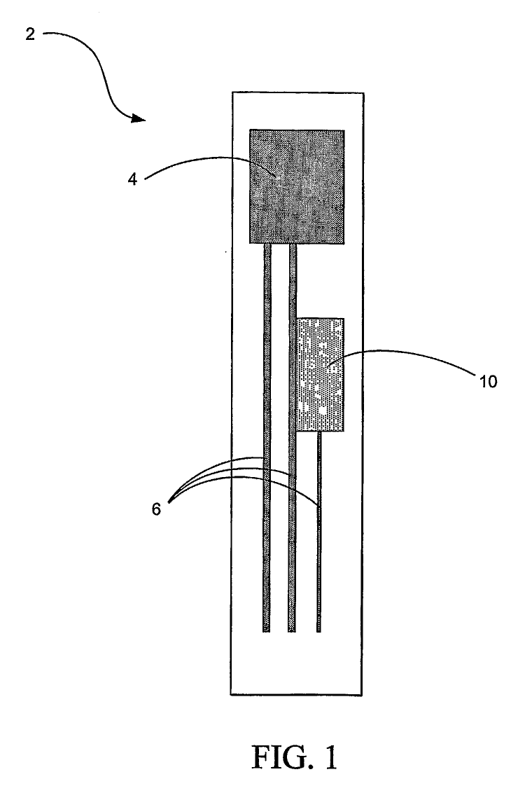

FIG. 1 shows a test element strip or test strip 2 having a sample area 4, electrical tracks 6, and a Radio Frequency Identification (RFID) tag 10. RFID (Radio Frequency Identification) is a technique which is able to carry data in suitable transponders, generally known as tags, and to retrieve data, by machine-readable means, at a suitable time and place to satisfy particular application needs.

An example RFID system may have, in addition to at least one tag, a transceiver or means of reading or interrogating the tags and optionally means of communicating the data received from a tag to an information management system. Transceivers are also known as interrogators, readers, or polling devices. Typically the system may also have a facility for entering or programming data into the tags. RFID tags contain an antenna and an integrated circuit. Various configurations of RFID tags are currently available in the marketplace and one such supplier is Texas Instruments® and the RI-I11-112A tag.

Communication of data between tags and a transceiver is by wireless communication. Such wireless communication is via antenna structures forming an integral feature in both tags and transceivers. During operation, the transceivers transmit a low-power radio signal, through its antenna, which the tag receives via its own antenna to power an integrated circuit. Using the energy it gets from the signal when it enters the radio field, the tag briefly converses with the transceiver for verification and the exchange of data. Once the data is received by the reader it is sent to a controlling processor in a computer for example, for processing and management.

RFID systems have pre-defined distance ranges over which tags can be read, which depend on several factors such as size of the antenna in the tag, size of the antenna in the transceiver, and the output power of the transceiver. Typically, passive RFID tags operate in the 100 KHz to 2.5 GHz frequency range. Passive RFID tags are powered from the transceiver, whereas active RFID tags have a power source such as a battery, which powers the integrated circuit.

Data within a tag may provide identification data for an item in manufacture, goods in transit, a location, the identity of a vehicle, an animal or individual. By including additional data the tags can support applications through item specific information or instructions immediately available on reading the tag. For example, the colour of paint for a car body entering a paint spray area on the production line, or the diabetes testing requirements of an individual e.g. on polling of the tag on the first test strip of the day, a user can be informed by the meter that he requires a further three glucose measurements during the next 24 hours.

Transmitting data is subject to the influences of the media or channels through which the data has to pass such as the air interface. Noise, interference and distortion are sources of data corruption that arise in the communication channels that must be guarded against in seeking to achieve error free data recovery. To transfer data efficiently via the air interface that separates the two communicating components requires the data to be modulated with a carrier wave. Typical techniques for modulation are amplitude shift keying (ASK), frequency shift keying (FSK) or phase shift keying (PSK) techniques.

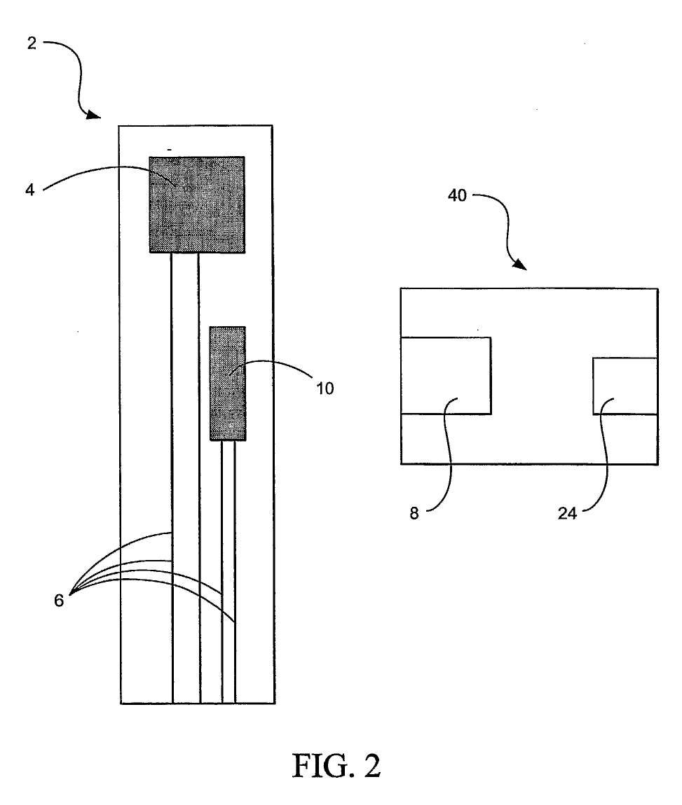

FIG. 1 shows a schematic plan view of test strip 2 of an auto calibration system as will be described hereinafter. Typically test strip 2 may be sized or shaped to fit into a slot on a meter 40 (see FIG. 2). The strip consists of an area 4 within which a patient's blood or ISF interacts with bio-reactive elements e.g. enzymes. This reaction causes a change in current on the conductive tracks 6 which is measured. The conductive tracks 6 may be configured to switch the meter on during insertion as will be described hereinafter. The meter 40 contains a means such as a transceiver including an RF source for polling or communicating with RFID tags. RFID tag 10 is fixed to the test strip 2 by means of pressure sensitive or heat seal or cold cure adhesive or alternatively printed on test strip 2 using e.g. carbon tracks during the manufacturing stage of the strip 2. For example, a coil in the RFID tag may be printed by screen printing a conductive track e.g. carbon, gold, silver in the form of a coil. The RFID tags can be written with calibration data, batch number, and expiry data or other data using RF encoding means after the strip has been manufactured.

The RFID tag can be placed in line on the tracks 6 so that during initial insertion the current also activates the RFID tag to cause it to transmit. Alternatively or in addition the RFID tag can be polled by exciting the tag via the transceiver both when the strip is in the meter and when the strip is not in the meter.

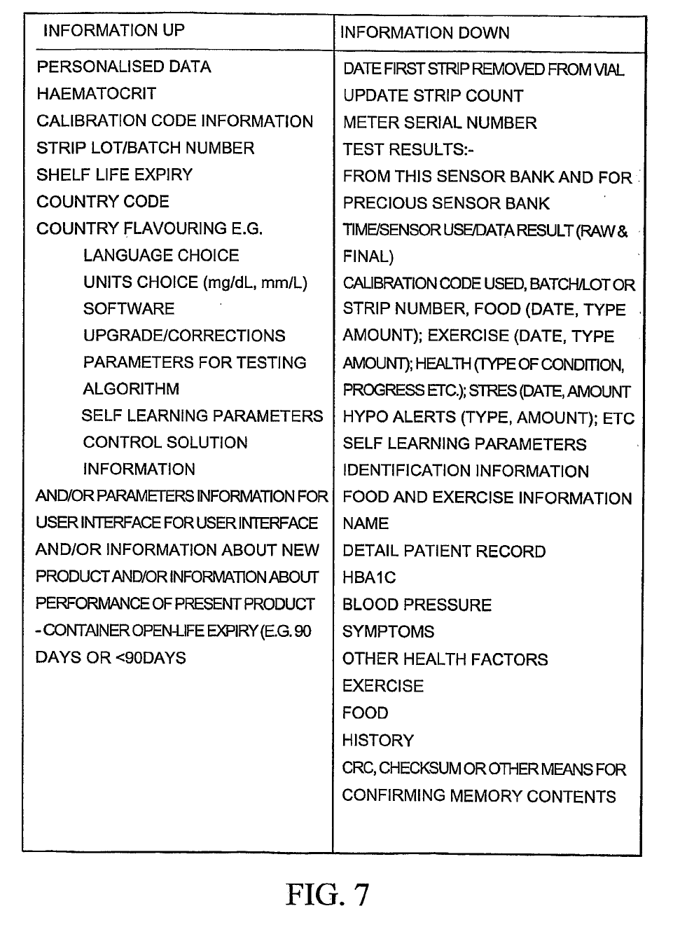

Referring to FIG. 1, the operation of a first embodiment of the invention will now be described in more detail. The single use test strip 2 has an RFID tag 10 containing information pertaining to batch number, and/or specific calibration data, and, optionally, other information such as ‘expiry date of strips’ information. Examples of information which can be obtained in an RFID tag in any of the embodiments of the invention is shown in the table in FIG. 7. Optionally, before inserting the strip 2 into the meter, the user of the meter activates the meter to a pre-fully functional mode for example by pushing a button. When in this mode, the meter polls for the RFID tag 10 on the nearest test strip. Alternatively, the strip 2 is inserted and the meter switched on (by strip insertion to close a contact or otherwise). The strip 2 may also activate the meter on insertion into the strip port connector 8, 18 by using a conductive track 6 on the strip 2 which forms a bridge between two conductors inside the meter itself. Once the meter is switched on it polls wirelessly for the RFID tag 10 closest to its transceiver. Thus, the RFID tag 10 on the test strip transmits the encoded information such as calibration information and/or batch number and/or expiry date and/or other information as described herein to the meter. Alternatively the tag 10 can be read via RF whilst the strip is in meter.

In an example system according to a first embodiment there is a meter and disposable test strip 2. The system containing a proximity interrogation system including a transceiver, a transponder (an RFID tag), and data processing circuitry. The transceiver includes a microprocessor, a transmitter, a receiver, and a shared transmit/receive antenna. The tag 10 is typically passive (having no on-board power source, such as a battery) and includes an antenna typically configured as a coil, and a programmable memory. As the tag 10 receives its operational energy from the reader, the two devices must be in close proximity. In operation, the transceiver generates sufficient power to excite the tag.

The polling for the RFID tag can either be continuous or activated by the user to enter a pre-fully functional status. When RF energy emanating from the reader's antenna impinges on the tag while it is in close proximity to the tag, a current is induced in the coil of the antenna. The tag does not need to be in line-of-sight of the meter and can typically operate in the range of a few centimetres or up to a few meters in circumstances as will be understood by persons skilled in the art. Alternatively, a transceiver having an antenna in a form of an array could be utilised which would increase the effectiveness of polling of the tag by increasing the angular range of communication. The induced current in the coil of the antenna is routed to the programmable memory of the tag, which then performs an initialization sequence. The transceiver transmits its energy transmitting interrogation signal to the tag and the memory in the tag begins to broadcast its identity and any other requested information over the tag antenna. Information transmitted to the transceiver is decoded as described below.

The transceiver in the meter, picks up the signal from the RFID 10 tag and the transmitted data is used in the processing of the test strip. Circuitry in the meter decodes and processes information received from the RFID tag 10. The strip 2 is inserted into a port 8 on a meter. A user lances a suitable site for example a finger or forearm or palm, and deposits blood or ISF on the sample area 4 on the strip 2. A measurement is made by the following method for example. A voltage is applied to test sensors within sample area 4 on the strip 2 and a current measurement is made. Calibration data is received from the tag 10 specific to strip 2 and is used for calculating the blood glucose level. This level is communicated to the user on the meter display.

The meter can optionally record when the first strip of that container is used. This can be used to calculate information for informing the user how long the vial has been opened, and if a use is recorded each time a strip is used, how many strips remain in a vial or cartridge. Thus, the circuitry in the meter can record the number of strips in a vial from strip information from the tag and then subtracts one from this number every time a strip is used from a specific batch of strips. This information combined with the batch number can be useful for a diabetic to either request additional strips from his physician or to calculate how fast a vial of strips is used over a period of time.

In case the RFID tag becomes damaged during the manufacturing process or during the transit to, e.g. the user, and cannot be read by the meter, or the battery level of the meter is too weak to poll for the RFID tag, the meter has circuitry for allowing a direct manual input of the calibration code. Indeed such direct manual entry can be provided as an option in any event. Typically, the calibration code would be printed on the side of the vial and the user could enter the calibration code before testing commenced. This would allow the user to continue using the strips, thus avoiding having potentially to discard a batch of strips because of a lack of calibration information due to a problem with the RFID tag.

FIG. 2 shows a test strip 2 having a sample area 4, conductive tracks 6, an RFID tag 10, and a meter having a strip port connector 8, and a wireless transceiver 24.

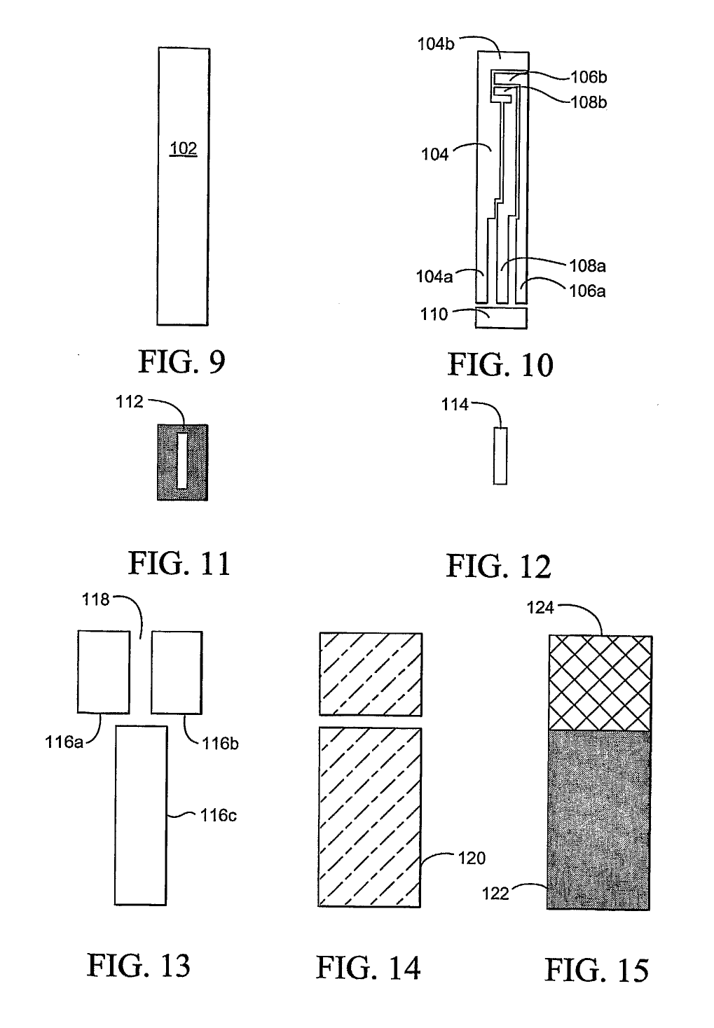

Generally speaking, the structure of the strip will be as follows. FIG. 9 shows an oblong polyester strip 102 which forms the base of a test strip for measuring the concentration of glucose in a sample of blood. The base member 102 is shown in isolation although in practice an array of such strips is cut out from a large master sheet at the end of fabrication. FIG. 10 shows the pattern of carbon ink which in this example is applied to the base member by screen printing, although any suitable deposition technique known in the art could be used. The layer of carbon comprises four distinct areas which are electrically insulated from one another. The first track 104 forms, at the distal end thereof, an electrode 104b for a reference/counter sensor part. The track 104 extends lengthwise to form a connecting terminal 104a at its proximal end. The second and third tracks 106, 108 form electrodes 106b, 108b at their distal ends for two working sensor parts and respective connecting terminals 106a, 108a at their proximal ends. The fourth carbon area is simply a connecting bridge 110 which is provided to close a circuit in a suitable measuring device to turn it on when the test strip has been properly inserted. These carbon areas, or other carbon areas printed at the same time can be shaped to provide a micro-strip antenna. Other carbon areas may provide a coil antenna or other component of a wireless device.

FIG. 11 shows the next layer to be applied also by screen printing. This is a water insoluble insulating mask 112 which defines a window over the electrodes 104b, 106b, 108b and which therefore controls the size of the exposed carbon and hence where the enzyme reagent layer 114 (FIG. 12) will come into contact with the carbon electrodes. The size and shape of the window are set so that the two electrodes 106b, 108b have a patch of enzyme of virtually the same area printed onto them. This means that for a given potential, each working sensor part in a batch will theoretically, and subject to accurate calibration, pass virtually the same electric current in the presence of a sample of blood.

An enzyme layer, in this embodiment a glucose oxidase reagent layer 114 (FIG. 12), is printed over the mask 112 and thus onto the electrodes 104b, 106b, 108b through the window in the mask to form the reference/counter sensor part and the two working sensor parts respectively. A 150 micron layer of adhesive is then printed onto the strip in the pattern shown in FIG. 13. This pattern has been enlarged for clarity as compared with the previous figures. Three separate areas of adhesive 116a, 116b, 116c together define a sample chamber 118 between them.

Two sections of hydrophilic film 120 (FIG. 14) are laminated onto the distal end of the strip and are held in place by the adhesive 116. The first section of film has the effect of making the sample chamber 118 into a thin channel which draws liquid into and along it by a capillary action. The final layer is shown in FIG. 15 and is a protective plastic cover tape 122 which has a transparent portion 124 at the distal end. This enables a user to tell instantly if a strip has been used and also assists in affording a crude visual check as to whether enough blood has been applied.

An RFID tag may be applied to the strip at any appropriate stage in its manufacture, and optionally after the application of the reagent layer. Applying the RFID tag to the strip before the protective plastic cover tape 122 will encapsulate the RFID tag and the RFID tag may simply be secured by adhesive, which may be conductive adhesive if and where the RFID tag makes contact with the electrodes or other deposited electrical components. It is better to select an adhesive with minimal outgassing characteristics. Optionally the tag may be adhered using the same adhesive used to secure the hydrophilic film, such as that used in the ONE TOUCH® Ultra Test strips available from LifeScan, Inc., Calif.

Further details of the strips, but not the use of RFID tags, can be found in international patent application no. WO 01/67099, which it would be pointless here to recount. Instead, the entire contents of WO 01/67099 are herein incorporated by reference.

As mentioned above, strips may be manufactured in a flat-bed or staged process in batches. In this process, electrochemical sensors are formed as a series of patterned layers supported on a substrate. Mass production of these devices has been carried out by screen printing and other deposition processes, with the multiple layers making up the device being deposited seriatim in a flat-bed process.

Manufacture of disposable electrochemical sensors by these techniques has several drawbacks. First, operation in flat-bed or staged mode is fundamentally inefficient. Multiple steps in the process requires the use of multiple flat-bed print lines, one for each layer in the device. Not only does this increase the capital expense for the manufacturing equipment it also introduces multiple opportunities for process variation such as variable delays and storage conditions between print steps, as well as variations in the process itself such as registration drift between different process stations. Such process variations can result in poor calibration of some sensor batches resulting in potentially erroneous reading when the electrodes are used. Variable delays and storage conditions may result, for example, in variable amounts of moisture being absorbed by the partly-manufactured sensors. The moisture content of the sensor is another example of a calibration quantity of the sensor.

A suitable method for manufacturing electrochemical sensors uses a continuous web of substrate transported past a plurality of printing stations for deposition of various layers making up the sensor. The method can be used for making sensors which are directed to any electrochemically-detectable analyte. This process still manufactures batches of sensors, with the size of the batch run typically being determined by the availability of consumables, especially the amount of substrate material available on a single roll. The remaining bulk and liquid components can be made available in the required quantities to use up a whole roll of substrate material.

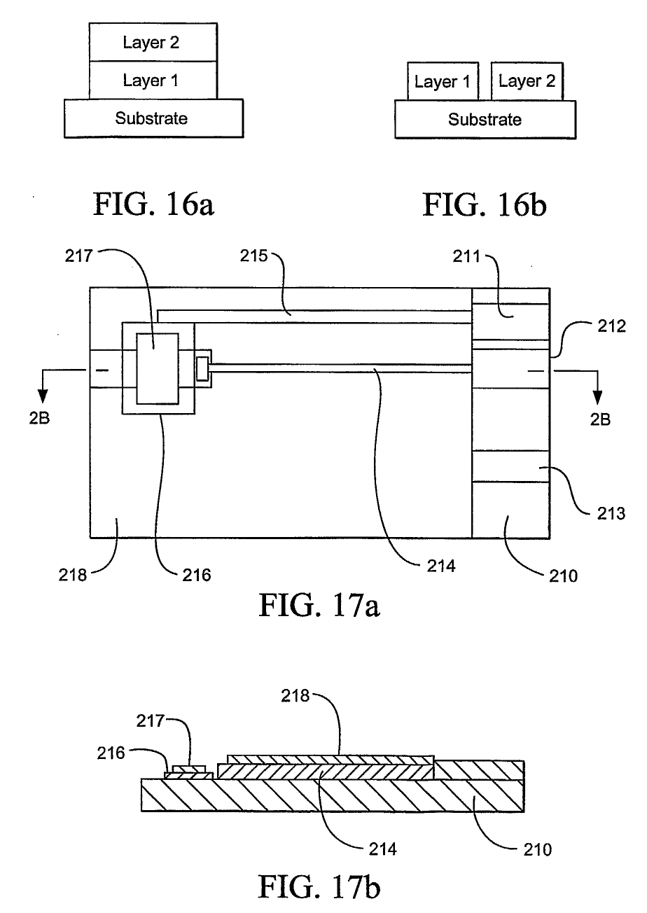

Exemplary analytes of particular commercial significance for which sensors can be made using the method include; glucose, fructosamine, HbA1C, lactate, cholesterol, alcohol and ketones. The specific structure of the electrochemical sensor will depend on the nature of the analyte. In general, however, each device will include an electrode layer and at least one reagent layer deposited on a substrate. As used in the specification and claims hereof, the term “layer” refers to a coating applied to all or part of the surface of the substrate. A layer is considered to be “applied to” or “printed on” the surface of the substrate when it is applied directly to the substrate or the surface of a layer or layers previously applied to the substrate. Thus, deposition of two layers on the substrate may result in a three layer sandwich (substrate, layer 1, and layer 2) as shown in FIG. 16A or in the deposition of two parallel tracks as shown in FIG. 16B, as well as intermediate configurations with partial overlap.

In the method of the invention, the electrochemical sensors are printed in a linear array, or as a plurality of parallel linear arrays onto a flexible web substrate. As discussed below, this web may be processed by cutting it into ribbons after the formation. As used in the specification and claims of this application, the term “ribbon” refers to a portion of the printed web which has been formed by cutting the web in either or both of the longitudinal and transverse directions, and which has a plurality of electrochemical sensors printed on it.

FIGS. 17A and 17B show the structure of an electrochemical sensors for detection of glucose in accordance with in the invention. On the substrate 210 are placed a conductive base layer 216, a working electrode track 215, a reference electrode track 214, and conductive contacts 211, 212, and 213. An insulating mask 218 is then formed, leaving a portion of the conductive base layer 216, and the contacts 211, 212 and 213 exposed. A reagent layer of a working coating 217, for example a mixture of glucose oxidase and a redox mediator, is then applied over the insulating mask 218 to make contact with conductive base layer 216. Additional reagent layers can be applied over working coating 218 if desired. For example, the enzyme and the redox mediator can be applied in separate layers.

It will be appreciated that the specific structure shown in FIGS. 16A and 16B is merely exemplary and that the method of the invention can be used to manufacture photometric, electrochemical or other sensors for a wide variety of analytes and using a wide variety of electrode/reagent configurations. Exemplary sensors which could be manufactured using the method of the invention include those disclosed in European patent no. 0 127 958, and U.S. Pat. Nos. 5,141,868, 5,286,362, 5,288,636, and 5,437,999, which are incorporated herein by reference.

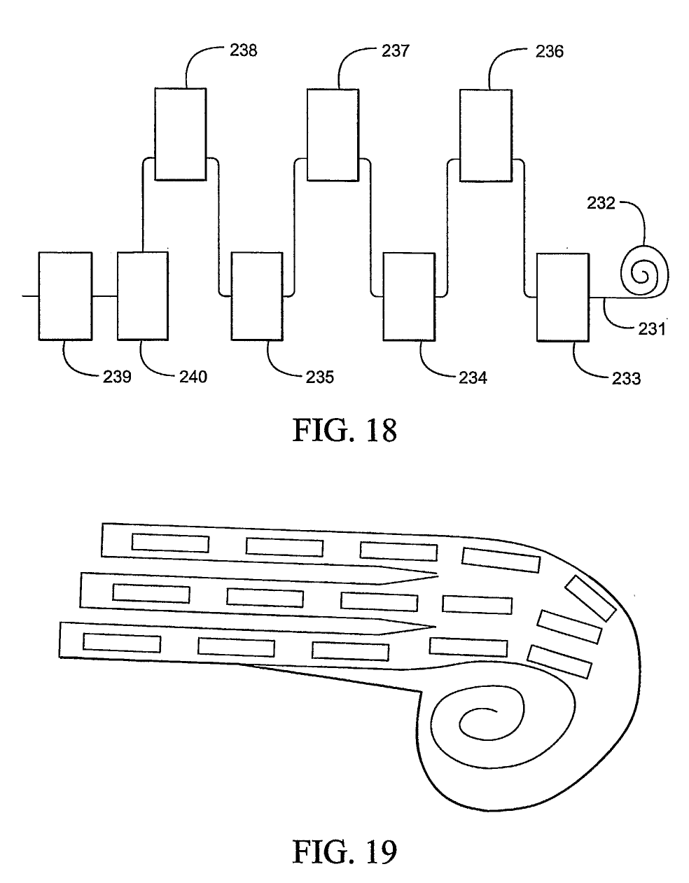

FIG. 18 shows a schematic view of an apparatus for practicing the invention. A running web of substrate 231 is provided on a feed roll 232 and is transported over a plurality of print stations 233, 234, and 235, each of which prints a different layer onto the substrate. The number of print stations can be any number and will depend on the number of layers required for the particular device being manufactured. Between successive print stations, the web is optionally transported through a dryer 236, 237, and 238 (for example a forced hot air or infra-red dryer), to dry each layer before proceeding to the deposition of the next. After, the final dryer 238, the printed web may be passed through an RFID fixing station 240 at which an RFID may be adhered to the structure using insulating or conductive adhesives as the case may be. Then it may be collected on a take up roll or introduced directly into a post-processing apparatus 39.

While the most efficient embodiments of the invention will generally use a plurality of print stations as illustrated in FIG. 18 for the printing of different materials, it will be appreciated that many of the advantages of the invention can be achieved with a process in which a single print station is used several times with different print reagents. In particular, benefits of increased throughput and improved print registration are obtained when using the same print station multiple times. Thus, we contemplate embodiments in which two or more distinct print stations are employed and embodiments in which a common print station is used in several passes or like print stations used in series to print the required materials onto the substrate.

One of the most important parameters to control when printing the various layers of a biosensor is the thickness of the deposited layer, particularly with respect to the reagent layer. The thickness of the printed layer is a calibration quantity of the sensor and is influenced by various factors, including the angle at which the substrate and the screen are separated. In a conventional card printing process, where the substrate is presented as individual cards on a flat table, this angle varies as the squeegee moves across the screen, leading to variations in thickness and therefore to variations in the sensor response across the card. To minimize this source of variation, the print stations used in the method of the exemplary embodiments optionally makes use of cylinder screen printing or rotogravure printing. In cylinder screen printing, a flexible substrate is presented to the underside of a screen bearing the desired image using a cylindrical roller and moves synchronously with the squeegee. Unlike conventional printing, where the screen moves away from a stationary substrate, in this process the moving substrate is pulled away from the screen. This allows a constant separation angle to be maintained, so that a uniform thickness of deposit is achieved. What is more, the contact angle, and thus the print thickness can be optimized by choosing the appropriate point of contact. By appropriate optimization, the process can be engineered so that the ink is pulled out of the screen and transferred to the substrate much more efficiently. This sharper “peel off’ leads to much improved print accuracy, allowing a finer detail print. Therefore smaller electrodes can be printed and smaller overall sensors can be achieved.

The post-processing apparatus 39 may perform any of a variety of treatments, or combinations of treatments on the printed web. For example, the post processing apparatus may apply a cover over the electrochemical devices by laminating a second continuous web to the printed substrate. The post-processing apparatus may also cut the printed web into smaller segments. To produce individual electrochemical devices of the type generally employed in known hand-held glucose meters, this cutting process would generally involve cutting the web in two directions, longitudinally and laterally. The use of continuous web technology offers the opportunity to make electrochemical sensors with different configurations which offer advantages for packaging and use.

As shown in FIG. 19, the printed web can be cut into a plurality of longitudinal ribbons, each one sensor wide. These ribbons can in turn be cut into shorter ribbons of convenient lengths, for example 10, 25, 50 or even 100 sensors. A short ribbon of say 5 strips can be prepared to provide enough sensors for one normal day of testing.

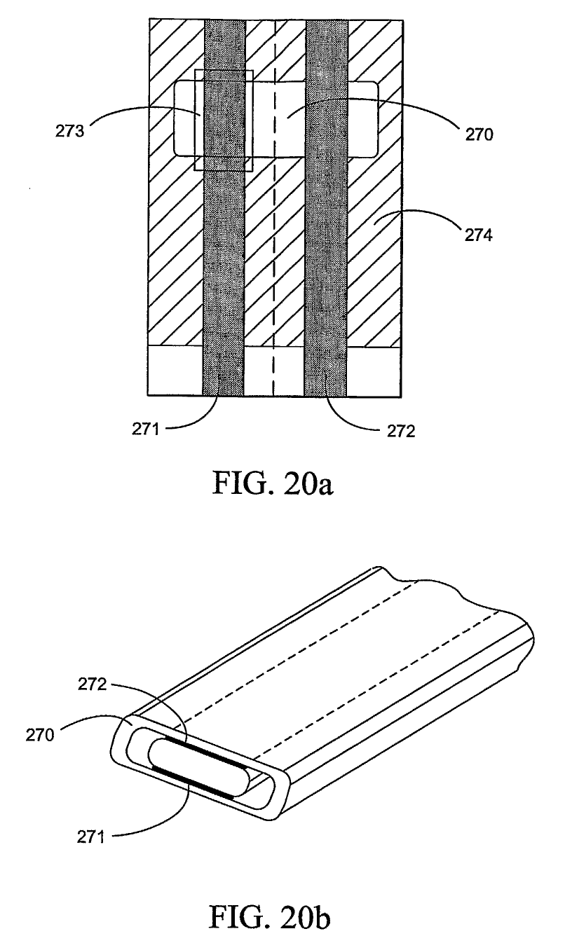

The method of the invention also facilitates the manufacture of sensors having structures which cannot be conveniently produced using conventional batch processing. For example, as shown in FIGS. 20A and 20B, a device can be manufactured by depositing parallel conductive tracks 271 and 272; reagent layer(s) 273 and an insulation layer 274 on a substrate 270. The substrate is then folded along a fold line disposed between the two conductive tracks to produce a sensor in which two electrodes are separated by a reagent layer. An electrode geometry of this type is beneficial because the voltage drop due to solution resistance is low as a result of the thin layer of solution separating the electrodes. In contrast, in a conventional devices with coplanar electrodes, the use of a thin layer of solution results in a substantial voltage drop along the length of the cell and concomitant uneven current distribution. Furthermore the device of FIGS. 20A and 20B can be cut across the deposited reagent to produce a very low volume chamber for sample analysis which further improves the performance of the device.

As is apparent from the foregoing discussion, the method of the exemplary embodiments provides a very versatile approach for manufacture and calibration of electrochemical sensors. The following discussion of suitable materials which can be used in the method of the invention is intended to further exemplify this versatility and not to limit the scope of the invention.

The substrate used in the method of the invention may be any dimensionally stable material of sufficient flexibility to permit its transport through an apparatus of the type shown generally in FIG. 18. In general the substrate will be an electrical insulator, although this is not necessary if a layer of insulation is deposited between the substrate and the electrodes. The substrate should also be chemically compatible with the materials which will be used in the printing of any given sensor. This means that the substrate should not significantly react with or be degraded by these materials, although a reasonably stable print image does need to be formed. Specific examples of suitable materials include polycarbonate and polyester.

The electrodes may be formed of any conductive material which can be deposited in patterns in a continuous printing process. This would include carbon electrodes and electrodes formed from platinized carbon, gold, silver, and mixtures of silver and silver chloride. Insulation layers are deposited as appropriate to define the sample analysis volume and to avoid a short circuiting of the sensor. Insulating materials which can be printed are suitable, including for example polyester-based inks.

The selection of the constituents of the reagent layer(s) will depend on the target analyte. For detection of glucose, the reagent layer(s) will suitably include an enzyme capable of oxidizing glucose, and a mediator compound which transfers electrons from the enzyme to the electrode resulting in a measurable current when glucose is present. Representative mediator compounds include ferricyanide, metallocene compounds such as ferrocene, quinones, phenazinium salts, redox indicator DCPIP, and imidazole-substituted osmium compounds, phenazine ethosulphate, phenazine methosulfate, pheylenediamine, 1-methoxy-phenazine methosulfate, 2,6-dimethyl-1,4-benzoquinone, 2,5-dichloro-1,4-benzoquinone, ferrocene derivatives, osmium bipyridyl complexes, and ruthenium complexes. Suitable enzymes for the assay of glucose in whole blood include glucose oxidase and dehydrogenase (both NAD and PQQ based). Other substances that may be present in the redox reagent system include buffering agents (e.g., citraconate, citrate, malic, maleic, and phosphate buffers); divalent cations (e.g., calcium chloride, and magnesium chloride); surfactants (e.g., Triton, Macol, Tetronic, Silwet, Zonyl, and Pluronic); and stabilizing agents (e.g., albumin, sucrose, trehalose, mannitol and lactose). The reagents appropriate to other types of sensors will be apparent.

It will be well understood that this structure causes the generation of both charge and current in the presence of an analyte, allowing for the following to be measured: an inter-electrode impedance; an inter-electrode current; a potential difference; an amount of charge; a change over time of any of the aforesaid; any combination of the aforesaid; or any other indicator of the amount of electricity passing from one electrode to another, or the extent to which exposure of the sensor to the fluid generates electrical energy or electrical charge or otherwise affects the electrical characteristics of the sensor.

One of the limitations of any device in which multiple test elements are stored within a test device is that the elements must be made stable for the expected lifetime of the test elements within the test device. In general, for electrochemical sensor strips, this means providing a moisture-proof and air-tight environment for unused sensor strips. This can be accomplished by adding a sealing layer to the test ribbon so that individual test strips are individually sealed and protected from moisture. Alternatively, one or more strips are contained in a vial such as that available from LifeScan, Inc. and sold as ONE TOUCH® Ultra.

Further details of the strips, but not the use of RFID tags, can be found in international patent application no. WO 01/73124, which it would be pointless here to recount. Instead, the entire contents of WO 01/73124 are herein incorporated by reference.

As discussed above, and seen in FIG. 2, the RFID tag 10 is fixed to the test strips and to electrodes or tracks 6 during manufacture. FIG. 2 shows a test strip 2 having a sample area 4, conductive tracks from the sample area 6 to an edge of test strip 2, and an RFID tag 10. A schematic of a typical meter is also shown which has a strip port connector 8 which is dimensioned to receive a strip 2. The meter also contains a wireless transceiver 24 which polls for information from the RFID tag 10. Conductive tracks emanate from the RFID tag to the edge of the test strip 2. Conductive tracks 6 to the RFID tag provide an additional mechanism for reading calibration data, expiry of strip data, batch number in the meter during use.

Photometric and calorimetric sensors can be manufactured in essentially similar processes or as described in U.S. Pat. No. 5,968,836, U.S. Pat. No. 5,780,304, U.S. Pat. No. 6,489,133, WO 04/40287 or WO 02/49507, the entire content of which are herein incorporated by reference. The RFID tag can simply be adhered to the finished strip or sensor, but is optionally positioned on the strip prior to the application of a protective layer.

Typical photometric or calorimetric sensor comprises a substrate and at least a first reagent including a catalyst and a dye or dye precursor and the catalyst catalyses, in the presence of the analyte, the denaturing of the dye or the conversion of the dye precursor into a dye. For glucose sensors, a suitable combination is a combination of glucose oxidase and horseradish peroxidase as a catalyst and leuco-dye as a dye precursor. The leuco-dye may, for example, be 2,2-azino-di-[3-ethylbenzthiazoline-sulfonate], tetramethylbenzidine-hydrochloride or 3-methyl-2-benzothiazoline-hydrazone in conjunction with 3-dimethylamino-benzoicacide. The reagent may be laid down as a film or membrane over a opening in a substrate or over a portion of a substrate or placed into a chamber in a substrate.

It is well understood that this combination of enzyme and leuco-dye causes the colour or depth of colour of the reagent layer to change in the presence of glucose, allowing for the following to be measured: opacity; transparency; transmissivity reflectivity or absorptivity; a transmission, reflection or absorption spectrum, peak, gradient or ratio; any one of more parts of such a spectrum; colour; a change over time of any of the aforesaid; and any combination of the aforesaid.

If a fluorophore is used instead of a non-fluorescing leuco-dye, the amount of glucose can be determined by looking at the fluorescence properties of the reagent, such as: fluorescence intensity; emissivity; an emission or excitation spectrum, peak, gradient or ratio; any one of more parts of such a spectrum; an emission polarization; an excited state lifetime; a quenching of fluorescence; a change over time of any of the aforesaid; or any combination of the aforesaid.

Returning now to FIG. 2, the application of the RFID tag 10 allows the calibration code data for each batch to be determined after the manufacturing process has been completed, i.e. after the constituent parts of the basic strip are in place. The RFID tags can be written with calibration data, batch number, and expiry data using RF encoding means after the strip has been manufactured.

During glucose testing, the diabetic inputs the test strip 2 into the meter. The diabetic lances himself and blood from his e.g. finger is drawn to the sample area of the strip. The meter is activated on insertion of the test strip 2 and current is applied to the reactive region of the strip. The meter either polls the RFID tag 10 for the calibration data, batch number, expiry date or alternatively the meter obtains calibration data, batch number, expiry date by using the tracks on the strip. This is a useful design feature of strips since if the meter has reduced power supply i.e. nearly life expired batteries or when a meter is being used in an RF noisy environment which may interfere with the polled RF signal transmission from and to the RFID tag, then the meter can still operate and obtain the calibration code for each batch of strips. Strips with an RFID tag hard wired or coupled through RF means, allows the user the option to check the validity of the calibration codes presented on the meter display or to cross check with calibration data presented on manufacturers' vials. Indeed, by producing both a hardwire connection to the RFID tag 10 and an RF connection to the RFID tag 10 from the meter, there is less scope for error in supplying the calibration code to the meter should one connection fail, or as a cross check.

The embodiments described can be used with integrated lancing/test strip devices such as those described in U.S. Pat. No. 6,706,159. When the meter is activated with the strip 2 inserted into the meter, the meter polls the RFID tag 10 for information specific to that strip 2 such as calibration code data and/or any other information as shown in FIG. 12. The data is then passed to the meter processor. A voltage is applied to the strip 2 and the current versus time data is read by the meter which calculates the glucose value. This glucose value is calculated using the calibration data and an algorithm or a combination thereof and then presented in the form of visual and/or auditory display.

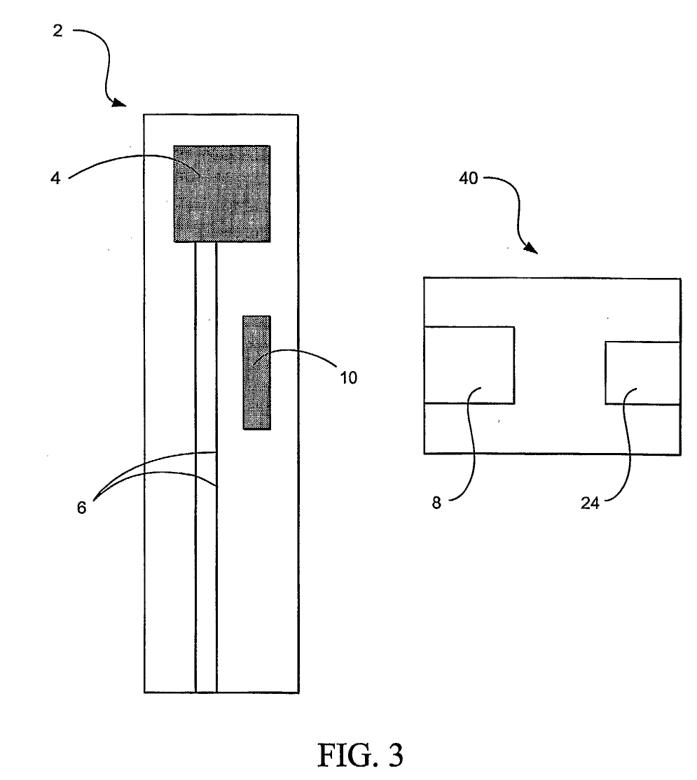

FIG. 3 shows a test strip 2 having a sample area 4, conductive tracks 6 from the sample area 4 to a short edge of test strip 2, and an RFID tag 10. A schematic of a typical meter is also shown which has a strip port connector 8 dimensioned to receive a strip 2. The meter also contains a wireless transceiver 24 which polls for information from the RFID tag 10, when the meter is activated. Meter activation is either by insertion of a test strip 2 as hereinbefore described or by manual depression of a button. Information is written to the RFID tag via RF after fixing of the tag to test strip 2.

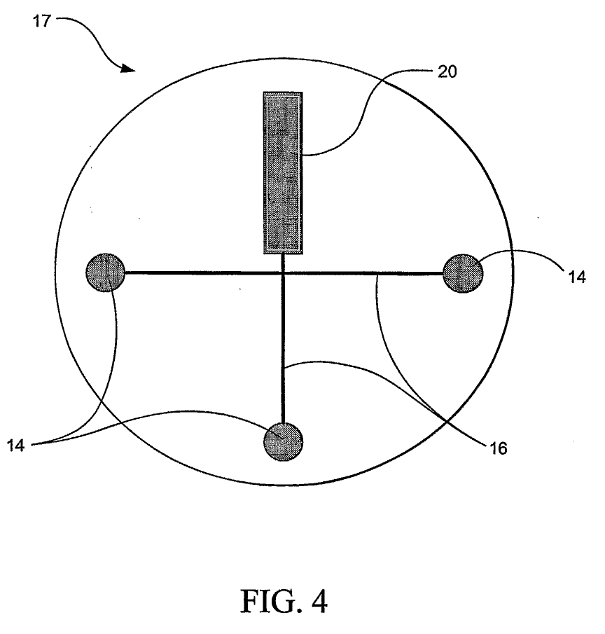

FIG. 4 shows a multi use test strip or module 12 in the form of a disc having three sample areas 14, conductive tracks 16, and an RFID tag 20. An RFID tag 20 is fixed to the test strip. The RFID tag can be activated to release information pertaining to calibration data and/or batch number and/or expiry of test strips 2 or other information as shown in FIG. 12 by providing a transceiver for example in a local controller or separate meter which transmits an appropriate RF field to activate the tag.

FIG. 5 shows a system 49 for extracting a bodily fluid sample (e.g., an ISF sample) and monitoring an analyte (for example, glucose) and includes a sampling device or cartridge (encompassed within the dashed box), a local controller module 44, and a remote controller module 43, a region of skin for sampling 47, a sampling module 46, and an analysis module 45.

Referring to FIGS. 4 and 5, a patient who controls his diabetes through continuous monitoring techniques would normally have a needle or similar attached to his skin. Blood or ISF is periodically or continuously pumped through the needle device to the continuous or multi use test strip 12 attached to the skin. In one embodiment, the continuous or multi use test strip 12 allows the diabetic to monitor his glucose levels without the daily repetitive lancing of his skin, which as previously discussed is a potentially limiting factor in testing due to several issues. Alternatively, the multi use module 17 (see FIG. 4), or array 27 (see FIG. 5) may be a used one strip 2 at a time by a user, the user having to produce (e.g. by lancing) a separate sample each time. These results may be used to give a quasi-continuous result composed of several discrete measurements.

Before use of the continuous or multi use test strip module 12 the patient applies the module to his skin. The module is fixed in place either using adhesive or adhesive strip or a strap. A small power source such as button cell is affixed to the sampling module 46. This button cell generates the voltage required for the reaction to take place and to provide an electrical signal to the meter. The current developed at the sensor region 14, 24 in multi-use module 17, 27 is measured by the local controller 44. Once the local controller 44 has measured has measured the current, or the current versus time data, the local controller 44 polls a tag on the test module to obtain, typically at least calibration code information. Using the measured data and the calibration code data the local controller 44 calculates the glucose level. The local controller 44 would typically be attached to the diabetic on his belt. The current or current versus time data is sent to the meter via a cable or via RF. For example the power source can also power a small transmitter in the local controller module 44 as well as the test strip 17, 27.

The user is informed of the glucose reading optionally initially through a vibration alert device and then through traditional notification means such as LCD display, sound alerts, voice alerts, or Braille instruction or a combination of these or simply through an audio alert and then a visual display.

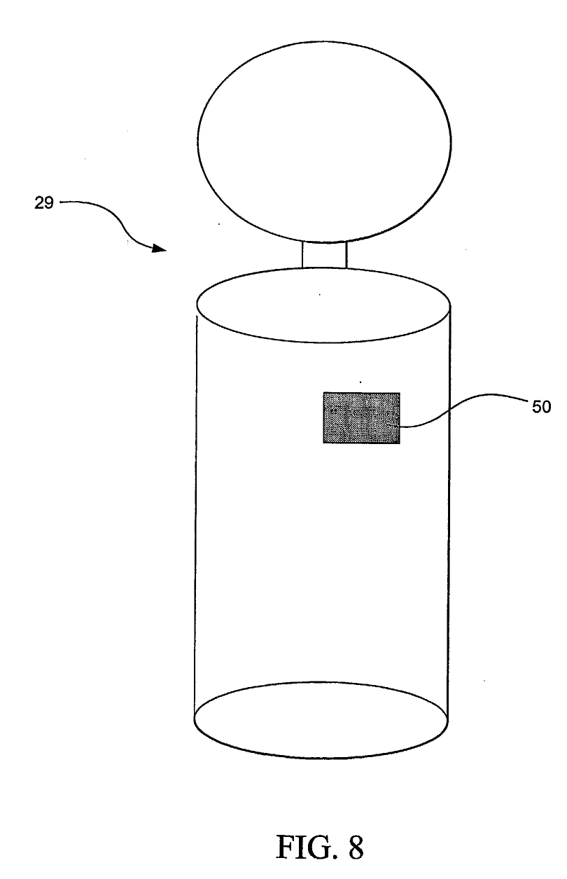

A vial 29 as shown in FIG. 8 may be used for storing test elements for testing for blood glucose for example. The vial 29 has a desiccant insert and good sealing lid and is used for containing strips 2. Such a vial 29 is available from Lifescan Inc (CA. USA) containing 25 ONE TOUCH® Ultra test strips. The utilization of the embodiments described herein is equally applicable to vials containing one or more test strips and to vials adapted to dispense test strips either within a meter or completely separately to a meter. For example U.S. patent application Ser. No. 10/666154 and EP 1, 518, 509 describe an integrated test element and lancet stored singly within individual vials (“microvials”), the entire content of which is herein incorporated by reference The utilization of the embodiments described herein is equally applicable to such a vial and integrated test element and lancet or even single test elements with no integral lancet. A dispensing test strip vial is described in U.S. patent application Ser. No. 10/081,368 and EP 1, 269, 173 “Test Strip Vial” and a dispensing strip vial within a meter is described in U.S. patent application Ser. No. 08/225,309 and U.S. Pat. No. 5,423,847 and U.S. patent application Ser. No. 10/880,145. The entire content of each of these documents is herein incorporated by reference.

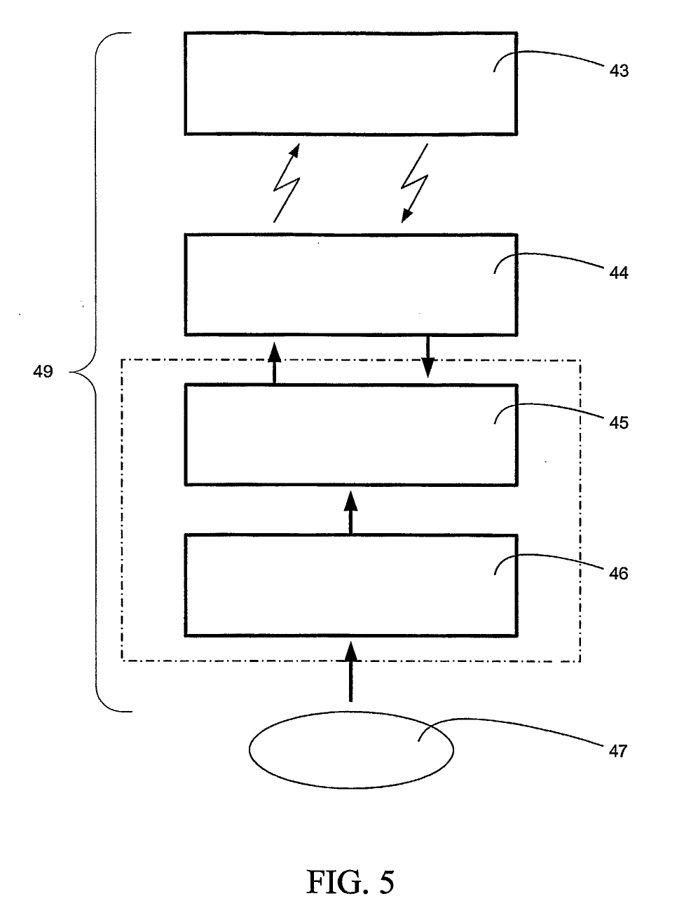

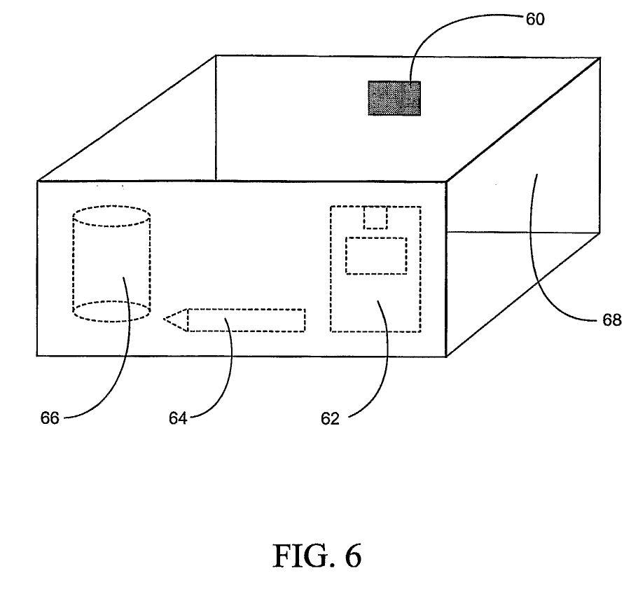

FIG. 6 shows a packaging container 68 containing a blood glucose meter 62, a vial 60 containing strips, an instruction booklet (not shown), a control solution bottle (not shown), and a lancing device 64. The packaging container has an RFID tag containing information such as calibration code, component identifier, batch identifier, manufacture identifier such as product code and/or packager, and/or manufacturer, and/or country of import/export, and/or language specific to country of import and/or a language sku containing reference to a number of languages e.g. American English and US Spanish or American English and Canadian French, and/or helpline specific to country of import, and/or product expiry date, and/or environmental storage conditions, and/or environmental conditions of use, and/or physiological limitations of use and/or other information as shown in FIG. 7. The RFID is programmed with such information after the contents of the packaging container have been ascertained from different suppliers e.g. vials and strips may be manufactured and packed at one factory, whereas the blood glucose meter may be manufactured at a different location and supplied from a different supplier. In other words, the consumer goods/final products are packed elsewhere and all individual items sent to a packaging factory for completion as a kit.

FIG. 7 details in addition to the information listed above the types of information which might be uploaded from an RFID tag to the meter and the types of information which might be written back down from the meter to the RFID tag for later use by a patient or clinician, or for use during further testing in any of the embodiments of the present invention.

The software of meters in the field may need to be upgraded and various embodiments described can be used to facilitate at least three types of changes. These are ‘corrective’—to fix problems, ‘adaptive’—to change the software in the light of changes to the environment in which the software runs (e.g. regulatory changes) and ‘perfective’—to change the software to add new features. A method of dynamically flavouring the meter with country code, personalised or country flavoured software, software upgrades and parameters related to previous test results for updating of the testing algorithm for future tests is also provided.

Referring now to FIG. 7, the operation of another aspect is described. Typically, when a user is initially diagnosed with diabetes, a physician will advise the diabetic that he needs to check his blood on a regular basis. One such system for blood glucose testing is the ONETOUCH® Ultra, manufactured by Lifescan. As described previously, most blood glucose meter systems use a test strip system which require entry of calibration code information into the meter on a per batch basis, periodic application of control solution, a meter which accepts the test strips, and a sample of blood obtained using a lancing device and applied to the test strips, which is inserted into the meter.

An RFID tag 60 is applied to the packaging container 68. During use, the diabetic retrieves the equipment required for a blood glucose test from the packaging container 68 and empties the contents, typically on a flat surface such as a table. The diabetic then follows a set procedure, guided by a display such as an LCD integrated on the meter 62. The meter 62 is activated either by insertion of the strip 61 or alternatively by manual pressing of a switch on the meter itself. Once activated, the meter 62 then polls for the RFID tag 60 located on the packaging container 68 and requests language option or country information such as country of import of product (e.g. a country or language sku), and product expiry date, environment storage conditions, and physiological limitations of use and/or calibration code. The information written into the RFID tag 60 on packaging container 68 is transmitted back to the transceiver on the meter 62. Such information is received by the blood glucose meter and transferred to a processor and into a memory card of the blood glucose meter. Information such as country of import obtained from the RFID tag 60, dictates which language is viewable on the LCD display e.g. for package containers intended for use in countries such as Germany, would have German user instructions (unless the user required another option). Similarly, in bi or tri-lingual countries such as Switzerland or Canada, the diabetic would have the option of specifying his language from within a range of those designated countries. Such an option is then subsequently programmed into the meter's memory and typically remains as the first option during an initial start up sequence and then becomes the default setting for any batch of strips i.e. further loading of RFID tag information from different vials or different packaging containers ignores data which contains language option information, in one embodiment of the invention the choice of language is used only during the initial start up of the blood glucose meter.

A useful feature of having such as a language option or a country specific code in the RFID tag 60, is that it allows the user to select a helpline facility specific to that country and language. Using the RFID country or language code from the RFID tag allows the diabetic to select helpline information for a country region which is most appropriate to the user. Indeed, a helpline registration system can be used so after initialisation of the meter using the first batch of strips the diabetic confirms his location and details to his regional supplier. The information held within the meter from the initial download of RFID tag 60 data could then be used to select country of normal residence. This user programmable data can either be activated by the diabetic following instruction from the manufacturers helpline number or using the instruction supplied on the screen, in his own language, and then saving this country code in the blood glucose meter 62.

When the next packaging 68 is used, the RFID tag on such packaging would relay country or language information to the meter on being polled by the meter. This information would be crosschecked with the country code embedded in the blood glucose meter's memory. If these are not the same, the meter would provide a message informing the diabetic that the meter will functioning temporarily and an incorrect test strip or batch may be used. On displaying such a stop message, the meter 62 displays a message or a warning message that the blood glucose meter needs to be reactivated by contacting the helpline. Indeed, a reset of the meter 62 can be performed. Typically, this can be performed through input of a numerical sequence or button pressing sequence available from the helpline facility. Such a reset procedure would also need the capability of needing a different sequence of numerical values or buttons pressing combinations for each reset, otherwise the user could simply reset the meter for each country or batch of strips each time, risking the use of inappropriate supply of strips. Such reset codes can be programmed into the meter memory during the manufacture thereof. The reset of the meter would not however be a total reset i.e. the patient's saved data would still be retrievable once successful reset code was input. As the RFID tag can contain more than one element of data, another useful element that can be sent to the meter at the first usage of a batch of strips, apart from the calibration code as previously described, is the provision of product expiry date and the number of test strips in a vial. Such information is useful for a diabetic and allows him to monitor the frequency he uses the test strips and/or the number of strips remaining. The numerical contents of the vial can be recorded in a memory of the meter obtained with information from the batch. Each time a test strip is used from that batch, the blood glucose meter records such usage and periodically, say every five test strips, informs the diabetic that he has used X strips and Y are left. Indeed, a higher frequency countdown can be implemented when the number of test strips in a vial is down to say 10. Such information can be displayed just after the next test strip is inserted requiring confirmation that the diabetic has understood the message or alternatively the message can be conveyed to the diabetic as a random message sent within a pre-defined time frame initially by vibration alert message followed by a standard displayed message. Again, the meter would again require confirmation by the diabetic that he has understood the message by button pressing or similar which would also switch off the repetitive nature of a vibration alarm system.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well. Finally, all publications and patent Applications cited in this specification are herein incorporated by reference in their entirety as if each individual publication or patent Application were specifically and individually put forth herein.

1-52. (canceled)

53. A method of making an analyte sensor, the method comprising:
providing a batch of analyte sensors;
affixing an RFID tag to each of the batch of sensors;
determining a calibration quantity for the batch of sensors; and
writing the calibration quantity specific to the batch of sensor to each of the RFID tag.

54. The method of claim 53, further comprising connecting electrically conductive tracks to the RFID tag.

55. The method of claim 53 in which the affixing comprises connecting the RFID tag to an electrochemical cell of the sensor.

56. The method of claim 55, in which the electrochemical cell comprises an electrode layer having at least two electrodes with a reagent disposed therebetween.

57. The method of claim 56, in which the reagent comprises an enzyme and an electron mediator material.

58. The method of claim 57, in which the sensor comprises a substrate on which the electrodes are formed thereon.

59. The method of claim 57, in which the reagent comprises glucose oxidase.

60. The method of claim 53, in which the sensor comprises an electrochemical sensor comprising electrodes configured to measure a characteristic selected from a group consisting essentially of an inter-electrode impedance; an inter-electrode current; a potential difference; an amount of charge; a change over time of any of the aforesaid; and combinations thereof.

61. The method of claim 53, in which the affixing comprises attaching the RFID tag to the sensor before the information representing the calibration quantity is transmitted to the RFID.

62. The method of claim 53, in which the providing comprises depositing an antenna layer on the sensor for the RFID.

63. The method of claim 62, in which the antenna comprises one of a micro-coil and micro-strip.

64. The method of claim 58, in which the providing comprises depositing an insulation layer over the electrode layer and depositing the reagent layer over the insulation layer, the insulation layer preventing contact between the electrodes and the reagent layer otherwise than at one or more selected contact zones.

65. The method of claim 64, in which the depositing comprises forming a mediator layer on the reagent layer.

66. The method of claim 53, in which the providing comprises forming a plurality of sensors on each sensor from a common batch.

67. The method of claim 66, in which each sensor comprises a sensor selected from a group consisting essentially of a colorimetric, photometric, and combinations thereof.

68. The method of claim 67, in which the sensor comprises a test strip.

69. A method of manufacturing an analyte sensor, the method comprising:
providing a continuous web substrate having a plurality of arrays on the substrate;
forming at least a conductive layer in each of the array, the at least one conductive layer including an antenna;
depositing reagent on each of the arrays of the substrate;
determining a calibration quantity of a sample of the plurality of arrays;
connecting a wireless transponder free of a power source to the conductive layer of each of the arrays of the substrate;
writing the calibration data to each wireless transponder of the plurality of arrays; and
forming an insulation layer over each of the arrays.

70. The method of claim 68, in which the depositing comprises defining a plurality of sensors on each array.

71. The method of claim 70, in which each sensor comprises a sensor selected from a group consisting essentially of photometric, colorimetric, and combinations thereof.

72. A method of using an analyte sensor, the method comprising:
affixing a wireless transponder free of a power source to the analyte test sensor;
writing data specific to the analyte test sensor to the wireless transponder;
energizing the wireless transponder via a remote transceiver; and
transferring the data from the wireless transponder to the remote transceiver.

73. The method of claim 72, in which the data comprises data selected from a group consisting essentially of calibration data, country, region, language, expiration date, batch number, storage condition, storage location, manufacturer, component code, and combinations thereof.

74. The method of claim 73, further comprising:
affixing the analyte sensor to the skin surface of a user;
measuring analyte quantity in a fluid sample of the user;
transmitting analyte data representative of the analyte quantity;
energizing the wireless transponder via the remote transceiver; and
conveying the analyte data to the remote transceiver.
